(19) United States
(12) Patent Application Publication
(10) Pub. No.: US 2003/0003137 A1
(43) Pub. Date: Jan. 2, 2003

(54) Method of applying frozen treatment chemicals to a fiber-based planar product and resulting products

(76) Inventors: Wolfgang Tissauer, Pernitz (AT); Peter Von Paleske, Seeheim-Jugenheim (DE)

Correspondence Address: YOUNG & THOMPSON, 745 SOUTH 23RD STREET 2ND FLOOR, ARLINGTON, VA, 22202

(21) Appl. No.: 10/169,138

(22) PCT Filed: Dec. 15, 2000
(86) PCT No.: PCT/EP00/12852

(30) Foreign Application Priority Data
Dec. 30, 1999 (DE) 199 63 834.9

(51) Int. Cl. A61K 9/70; D21H 23/00

(52) U.S. Cl. 424/443; 162/100

(57) ABSTRACT

The present invention relates to a method of applying a treatment composition to a fiber-based planar product, in particular tissue, said method comprising the steps of: a) producing particles from a frozen treatment composition containing at least one treatment chemical, c) applying the frozen particles of the treatment composition to the planar product, d) heating the frozen particles of the treatment composition to the melting point of the frozen treatment composition or to a higher temperature, as well as an apparatus usable for this method and a fiber-based planar product obtainable by this method. The method of the invention is particularly suitable for the application of treatment chemicals having chemical or physical properties which are difficult to bring into harmony, when applying these chemicals.

The present invention relates to a method of applying treatment chemicals to fiber-based planar products, particularly tissue. It also relates to a device for performing the method and to the products made using same, particularly tissue products.

Within the framework of the present invention, the term “tissue” especially includes “tissue paper” or “raw tissue”, as is normally produced as a one-ply tissue web in the tissue (paper) machine, as well as including multiply (intermediate) products, e.g. in the form of multiply doubled webs or in the form of master rolls for further processing and ready-made one-ply and multiply tissue products such as paper handkerchiefs, facials, toilet paper, household towels such as kitchen towels, hand towels and other wipes etc.

### PRIOR ART

Based on the underlying correspondence of the production processes (wet laying), “tissue” production is counted among the paper making techniques. The production of tissue, or more accurately, raw tissue if the one-ply (intermediate) product manufactured on a special-purpose paper machine of the tissue or tissue paper machine is meant, is delimited from paper production as a result of the extremely low basis weight of normally less than 40 g/m² and as a result of the much higher tensile energy absorption index as compared to paper. The tensile energy absorption index is arrived at by relating the tensile energy absorption to the test sample volume before inspection (length, width, thickness of sample between the clamps before tensile load).

Paper and tissue paper also differ in general with regard to the modulus of elasticity that characterizes the stress-strain properties of these planar products as a material parameter, depending on the production conditions, raw materials used and chemical additives.

A tissue paper's high tensile energy absorption index results from the outer and/or inner creping. The former is produced by compression of the tissue paper web adhering to a dry cylinder as a result of the action of a crepe doctor or in the latter instance as a result of a difference in speed between two successive screens or e.g. between a sheet-forming screen and a so-called fabric or between two fabrics.

When applying the through air drying (TAD) technique for the production of raw tissue and the usual double-screen sheet formation in c-wrap configuration, for example, the so-called inner sheet-forming screen can thus be operated at a speed that is up to 40% faster than that of the next fabric or that of the subsequent felt, the initially formed and already pre-drained paper web being transferred to the next TAD fabric. This causes the still moist and as a result plastically deformable paper web to be internally broken up by compression and shearing, thereby rendering it more stretchable under load than a paper that has undergone neither “internal” nor external creping.

This transfer of a still plastically deformable paper web at a differential speed that simultaneously takes effect may also be brought about in other embodiments between a transfer fabric and the so-called TAD imprinting fabric or between two transfer fabrics.

German has adopted the English-language term “fabric” to designate paper machine covers that exhibit a screen-like fabric structure in which synthetic threads are used as a thread material instead of metal wires.

Most of the functional properties typical of tissue and tissue products result from the high tensile energy absorption index (see German standards DIN EN 12625-4 and DIN EN 12625-5). An example is represented by tissue products for hygienic applications (hygiene products, particularly hygiene paper products) which are e.g. used in personal grooming and hygiene, the household sector, industry, the institutional field in a very wide variety of cleaning processes. They are used to absorb fluids, for decorative purposes, for packaging or even just as supporting material, as is common for example in medical practices or in hospitals. In terms of their wide variety, hygiene products are now considered to be everyday products.

Hygiene paper primarily includes all kinds of dry-creped tissue paper, as well as wet-creped paper.

The one-ply intermediate products originating from the paper machine and made of lightweight, i.e. low basis weight paper usually dry-creped on a yankee cylinder by means of a crepe doctor are generally described as “tissue paper” or more accurately raw tissue paper. The one-ply raw tissue may be built up of one or a plurality of layers respectively.

All one-ply or multiply final products made of raw tissue and tailored to the end user's needs, i.e. fabricated with a wide variety of requirements in mind, are known as “tissue products”.

Typical properties of tissue paper include the ready ability to absorb tensile stress energy, their drapability, good textile-like flexibility, properties which are frequently referred to as bulk (crumple) softness, a high surface softness, a high specific volume with a perceptible thickness, as high a liquid absorbency as possible and, depending on the application, a suitable wet and dry strength as well as an interesting visual appearance of the outer product surface. These properties allow tissue paper to be processed into tissue products (tissue paper products) and are then available to end users in a wide variety of forms and fabrication, for example as wipes, towels, household towels, particularly as kitchen towels, sanitary products (e.g. toilet paper), paper handkerchiefs, cosmetic tissues (facials) or serviettes/napkins.

Depending on the particular application, varied and to an extent conflicting properties are frequently needed for the successful use of tissue products in their extremely broad range of applications.

For this purpose, the tissue is frequently provided with substances, additives, auxiliary substances and other treatment chemicals.

In accordance with the invention, this term will also cover any substance or blends of substances generally referred to as treatment chemicals and normally applied to the tissue after the drying and creping step on the yankee cylinder.

Treatment chemicals may have an influence on physical properties, e.g. softness, particularly bulk softness, strength in the dry and wet states, rate of absorption of liquids, particularly that of water or oil, or the structural strength of the tissue/tissue product itself, and/or they may contribute to their varying use, e.g. in the field of skin care and protection, healthcare, etc. “Lotions” are also particularly referred to in the latter case.

Household towels for example, particularly kitchen towels and to an even greater extent paper towels, require strength, especially in the wet state, and high suction capacity so as to satisfy consumer demands. In the case of toilet paper, a combination of dry strength plus good softness is more likely to determine suitability in practice and acceptance among consumers. In the case of other tissue products such as handkerchiefs or facial wipes, surface softness and excellent suppleness are predominant properties which, in addition to strength, define the serviceability of these products.

Cosmetic components contained in the product and particularly present on its outer surfaces also play an important part in the latter tissue products. Such cosmetic components include, inter alia, perfumes, moisturizers, skin care agents, healthcare substances such as D-panthenol or the active camomile ingredient α-bisabolol.

It is important in the case of cosmetic components to achieve an optimum transfer of the components such as care agents or moisturizers from the tissue product to the skin—optimum in the sense of an adequate quantity of such components—so as to promote the desired effect. High amounts of the cosmetic substances to be applied to the tissue are necessary for this purpose. On the other hand, the tissue itself must not feel unpleasant or e.g. leave behind a wet feeling on the skin.

Manufacturers of tissue products are therefore especially faced with the challenge of achieving a particular balance between the various, frequently contradictory parameters in order to use this balance to obtain the optimum combinations of features required by consumers for the desired final products. The article entitled “Weichheit und Weichmachung von Hygiene-Tissue” in the Wochenblatt für Papierfabrikation, No. 11/12, 1988, pages 435 et seq., describes in detail the properties of hygiene tissue and discusses their importance to tissue products in different applications.

Thus, one of the principal market demands to be met by manufacturers is a general improvement in softness in all areas of tissue products. Properties such as the softness of a tissue product are defined in terms of their basic design by the production process, particularly by preliminary TAD and the choice of raw and auxiliary materials.

Softness is an important property of tissue products such as handkerchiefs, cosmetic wipes, toilet paper, serviettes/napkins, not to mention hand or kitchen towels, and it describes a characteristic tactile sensation caused by the tissue product upon contact with the skin.

Although the term “softness” is generally comprehensible, it is extremely difficult to define because there is no physical method of determination and consequently no recognized industrial standard for the classification of different degrees of softness.

To be able to detect softness at least semi-quantitatively, softness is determined in practice by means of a subjective method. To do so, use is made of a “panel test” in which several trained test persons give a comparative opinion.

In simplified terms, softness can be subdivided into its main characteristics, surface softness and bulk softness.

Surface softness describes the feeling perceived when e.g. one's fingertips move lightly over the surface of the sheet of tissue. Bulk softness is defined as the sensory impression of the resistance to mechanical deformation that is produced by a tissue or tissue product manually deformed by crumpling or folding and/or by compression during the process of deformation.

The application of the aforementioned treatment chemicals with which e.g. the desired softness characteristics or other properties are to be achieved is brought about in the prior art by different roll and spray application techniques. Other methods include impregnation techniques.

WO 94/05857 describes a method of applying a chemical paper-making additive to a dry tissue paper mat (tissue paper nonwoven fabric, raw tissue). The application technique is characterized by the following steps: provision of a dry tissue paper mat, dilution of a chemical paper-making additive using a suitable solvent to form a diluted chemical solution, the application of this diluted chemical solution to a heated transfer surface, partial evaporation of the solvent through the transfer surface to form a film that contains this paper-making additive and the transfer of this film from the heated transfer surface to the surface of the tissue mat.

EP-A-03 47 177 relates to a method of making soft tissue paper comprising the following steps: forming sheets from an aqueous suspension of cellulose fibers to form a mat, application of a sufficient amount of water-soluble non-cationic surfactant and drying and creping the mat, this tissue paper exhibiting a basis weight of 10 to 65 g/m² and a density of less than 0.6 g/m³.

The treatment solution can therefore be added both in the wet section of a tissue paper machine (wadding machine), at the end of the screen section, before or inside the press section (mechanical drainage), i.e. in the case of solid contents between 20 and 50%, and in the dry section disposed after the press section in the case of solid contents of 40 to 97% fibrous dry weight.

The prior art is represented by feed sites on the transfer screen/belt, e.g. ahead of mat transfer in a TAD layout, and the supply to the moist fibrous mat after its transfer to the transport (dry) felt before the press or presses in a conventional single-felt or double-felt tissue machine.

The supply of treatment chemicals by spray application onto the yankee cylinder is also known in the prior art.

The addition of the treatment agent within the tissue making machine is brought about by spray application onto the pope roller to produce a film of treatment agent and subsequently to transfer it to the tissue web during rolling up. The already creped “tissue web” usually still exhibits a residual temperature of between 20° C. and about 70° C. as a result of the preceding drying process on the yankee cylinder, which benefits the distribution of treatment agent and its penetration of the raw tissue.

In addition to spray application via a nozzle bar, the use of centrifugal rotors or brush units is possible. Application may also be effected directly onto the tissue paper web.

Addition of the treatment agent, within a so-called doubling machine or within the processing machine, to the outer plies of the multiply doubled web before or during calibration/smoothing is generally preferred.

Application of the treatment agent to the outer plies of the web frequently takes place within the processing machine, the web being guided in multiply fashion by use of a plurality of unwindings in the processing machine or being previously doubled in multiply fashion.

WO 98/41687 describes a method of making tissue products of the aforementioned kind, this method being characterized by the fact that a composition of the above type is applied to the fibrous mat or tissue web within the screen section, press section, TAD section, on the yankee cylinder and/or dry section, i.e. at a fibrous material density of 20 to 97%, relative to the web's dry fibrous weight, in an amount of 0.1 to 40%, preferably 1 to 20%, continuously or discontinuously on or within the web and the web may undergo post-smoothing after application.

An alternative embodiment mentioned in this document relates to a method of making tissue products, this method being characterized by the fact that a composition of the above type is applied to the fibrous mat or tissue web after the dry section on the wadding machine, doubling machine and/or in the automatic processor in an amount of 0.1 to 40 wt. %, preferably 1 to 20 wt. %, continuously or discontinuously on or within the web and the web may undergo post-smoothing after application.

The known techniques suffer from various disadvantages that lead to an impairment of the tissue properties. The pressure exerted on the tissue, e.g. when using roller application techniques to apply the treatment agent, particularly during follow-up smoothing of the product treated with a treatment agent, causes the occurrence of undesirable mechanical effects upon the tissue. The tissue is compressed, thereby decreasing e.g. its thickness (bulk), which consumers usually feel to be detrimental e.g. in the case of a paper handkerchief. Such a subjective impression on the user's part in the example of a thickness that is perceived to be detrimental may in turn wreck any objective improvement e.g. in surface softness, because consumers refuse to buy such a product. This is a problem that is particularly faced by multiply tissue products.

Roller or spray-on application is limited by the viscosity of the lotion to be applied. Highly viscous and/or fatty lotions can be applied to paper by means of a spray technique only with extreme difficulty or not at all. It is therefore often necessary to use e.g. water or organic solvents to dilute or refine the treatment agent to be applied, entailing another process step in which the employed solvent has to be removed from the tissue once more.

If treatment chemicals that exhibit high viscosity (or are solid) at room temperature or exhibit low viscosity at room temperature are to be applied to the tissue in combination, it is frequently hard to find equipment and application conditions that enable even and systematic application for both components.

A similar problem may arise in the case of hydrophobic (e.g. fatty) and hydrophilic components that tend to separate, which also makes even and systematic application difficult.

Lastly, the application of cosmetic treatment chemicals (sometimes also known as “cosmetic lotions”) to tissue products occasionally suffers from the problem that the cosmetic lotions completely penetrate the tissue, although they develop their action only upon transferral to the user's skin, which makes enrichment necessary on the external surface (or surfaces) of the tissue. For economic reasons, there is consequently a need for application processes that enable enrichment of cosmetic lotions on the external surface (or surfaces) of the tissue product.

An attractive application process must therefore simultaneously be able, for legal reasons, e.g. on grounds of competition law, to guarantee even application of the treatment chemicals on the external surfaces of the tissue product's outer plies, and on the other hand this process must, for reasons of economy, control within narrow limits the amount of such chemicals applied, because the cosmetic components of a lotion simultaneously represent very significant cost factors.

### OBJECT OF THE INVENTION

It is therefore an object of the invention to enable the simultaneous application of a plurality of treatment chemicals to a fiber-based planar product, particularly tissue, such chemicals exhibiting, in terms of application, chemical and/or physical properties that are not very compatible with each other.

Another object of the present invention is to make available an application process that is suitable for the enrichment of one or more treatment chemicals on the external surface or surfaces of a tissue or tissue product.

### DETAILED DESCRIPTION OF THE INVENTION

This object is solved by a method of applying a treatment composition to a fiber-based planar product, particularly tissue, such a method comprising the steps of

producing particles from a frozen treatment composition that contains at least one treatment chemical (henceforth also referred to as step (a)),

applying the frozen particles of the treatment composition to the planar product (henceforth also referred to as step (c)),

heating the frozen particles of the treatment composition that were applied to the planar product to the melting point of the treatment composition or a higher temperature (henceforth also referred to as step (d)).

The invention will be described in the following using the example of a tissue, but it is not restricted thereto and is applicable to any fiber-based planar product.

In a preferred embodiment of the method according to the invention, the treatment composition is frozen by bringing the treatment composition into contact with a fluid, i.e. liquid or gaseous medium, and hence converted into a solid state. The medium exhibits a temperature at or below which the treatment composition freezes. This preferred version of the method according to the invention comprises the steps of:

conversion of the treatment composition into the frozen state by bringing the treatment composition into contact with a fluid medium exhibiting a temperature at which the treatment composition exists in a frozen state (henceforth also referred to as step (a′)),

separation of the frozen treatment composition from the fluid medium, and optionally before or after this step, production of particles from the frozen treatment composition if this composition has not accumulated in particle form upon conversion into the frozen state (henceforth also referred to as step (b)),

application of the frozen particles of the treatment composition to the planar product, (step (c)),

heating the particles of the frozen treatment composition that were applied to the planar product to the melting point of the frozen treatment composition or a higher temperature (step (d)).

In step (a) or (a′), the frozen treatment composition may be produced for example in the form of fairly fine particles, fairly coarse particles (e.g. as coarse granules) or as a solid substance.

A solid substance can be produced for example by having it cooled, and preferably stirred, in a closed coolable container. This makes it possible to convert the treatment composition into the frozen state even without contact with a fluid medium. Depending on requirements, the solid substance may be converted into coarser or finer particles by means of comminuting techniques.

Finer particles are also obtained for example when the treatment composition, optionally after thoroughly mixing the constituents, is brought into contact, in a finely dispersed form (e.g. by spraying), with a cold medium at a suitable temperature. Particles are preferably produced by introducing the treatment composition into a liquid medium, it being possible to obtain a suspension of fine particles e.g. by vigorously stirring the medium or by its introduction in a finely dispersed form (particularly spraying). The temperature of the fluid medium is preferably so low that introduction of the treatment composition causes it to freeze immediately into solid particles. The amount introduced into the medium is chosen to be so low that the viscosity of the suspension is essentially determined by the viscosity of the fluid medium. The latter is preferably low and usually ranges from 1.0 to 0.1 mPas. Liquid nitrogen as a fluid medium has a viscosity of 0.2 mPas. The particle size and its distribution can be arbitrarily controlled by suitable selection of the spray nozzles. Effects of shearing forces (e.g. by means of an Ultraturrax) for further precise adjustment of the particle size are also possible, as is the use of screens. In the case of high-viscosity treatment compositions, the storage containers and feed lines for the treatment compositions as well as the spray nozzles can be heated.

Coarse particles, e.g. coarse granules, can be obtained using a device that functions e.g. like a laboratory ice-making machine, with the aid of receptacles, apparatus, insulation and deep-freezing equipment necessary for this purpose.

Inert media are particularly suitable as a fluid medium with which the treatment composition can be contact-frozen. In this context, inert means that the medium does not react with the active constituents, especially the treatment chemical(s), the treatment composition, and particularly does not dissolve them. Liquid carbon dioxide, which exhibits excellent dissolving properties for organic substances, is therefore normally out of the question as an inert medium.

The fluid medium should also be easily separable from the frozen particles, e.g. by screens, whereby the porous tissue web itself can be used or separation. Other criteria to bear in mind when selecting the medium are environmental compatibility and safe handling. The fluid medium is preferably nitrogen, air or a CFC or fluorohydrocarbon (also known as freons) present either in a (considerably) cooled gaseous form or in liquid form. For reasons of environmental protection, CFCs or fluorohydrocarbons are preferably recirculated within completely closed systems.

The temperature necessary for freezing the treatment composition can easily be determined by the skilled person by means of the melting points of the individual components or in experiments. The temperature is usually below the melting point of the component with the lowest melting point, but may also be above it, e.g. if this component accounts for just a very low proportion of the treatment composition. Depending on the freezing point, the following temperatures are particularly suitable for the majority of treatment compositions used in tissue making: below −25° C., preferably less than −50° C., greater preference being given to less than −75° C. Even greater preference may be given to temperatures of less than −100° C., particularly those between −200 and −100° C.

If a fluid medium is used for freezing, this medium has to be separated, before or during application to the planar product, particularly to tissue, from the frozen treatment composition (present e.g. as fine particles or as coarse granules) (separation, optional step b).

This may for example be brought about by means of a screen body that is suitable in terms of mesh width. Due to its production-related porous structure and its low basis weight, the planar product, particularly tissue, may itself be used to separate the particles, the steps of separation (b) and application (c) then being performed simultaneously.

After its separation from the frozen treatment composition, the medium is preferably reprocessed and optionally recirculated.

Before application (step (c)), it may be beneficial to generate a suitable particle size distribution of the frozen treatment composition. This can also be performed separately for the treatment composition's various constituents, the particle fractions obtained then being suitably mixed before application.

Thus the recovery of coarse particles (e.g. coarse granules) of the treatment composition makes it preferable to comminute these particles either between steps (a)/(a)′ and (b) or between steps (b) and (c), or, if no step (b) is applied, between steps (a) and (c), optionally followed by a classifying step, so as to achieve a particle size distribution that is ideal for application. A comminuting step, optionally with subsequent classification, is also preferred in the case of frozen treatment compositions in the form of solid substances. If the frozen treatment composition is already present in the form of fine particles, a further comminuting step is usually unnecessary, thus making direct classification possible.

Comminution of the frozen treatment composition may for example by effected by the influence of mechanical energy, such as the influence of mechanical pressure upon the coarse granules in the roll gap between two or more rolls or by grinding between the plates of a refiner. The means of comminution (e.g. roll(s) or refiners) are preferably cooled in order to prevent the frozen treatment composition from thawing early. Ultrasound can also be used to comminute the particles.

For the purpose of classifying, conventional classifying means such as screens or classifiers (e.g. centrifugal classifiers) are suitable; these are preferably cooled to prevent the frozen treatment composition from thawing prematurely.

Before application, it is also possible, depending on the desired composition, to blend one or more particle size fractions of the treatment composition (or its constituents). This may, for example, be brought about by using a turbulence generator, e.g. a mechanical stirring device, to mix the frozen particles of the individual components of the treatment agent, these particles being present in a deep-frozen, fluid inert medium in suspended form in a deep-freezable mixing receptacle. Mixing preferably occurs in the absence of the fluid medium.

Upon application (step c), the particles of the frozen treatment composition are applied to the planar product, especially tissue. The frozen treatment composition is preferably applied to one or both external surfaces of a tissue (single-ply or multiply). The deep-frozen treatment composition behaves “inertly” , i.e. it does not exhibit the physical/chemical properties of its individual constituents (i.e. treatment chemicals), particularly not its viscosity. This easily permits even distribution of the treatment composition over the surface dimension of the planar product, particularly tissue, in just one operating cycle using a single applicator, even though there may be very different physical properties at room temperature as regards the individual components of the treatment composition, e.g. in terms of their viscosity, which would have made it necessary to use a variety of different application units in succession in the case of application at room temperature. The method according to the invention thus makes it possible to manufacture tissue products which in addition to constituents exhibiting low viscosity at room temperature contain e.g. constituents that are highly viscous (or even solid), pasty or fatty at room temperature.

The frozen treatment composition is preferably applied by evenly “trickling” deep-frozen fine particles of the treatment composition. The frozen state allows the particulate treatment composition to trickle.

The application of frozen particles using a single application step preferably occurs by trickling the powdery, deep-frozen granular material of the treatment agent onto the fiber-based planar product, e.g. the moving tissue web (this powdery material being preferably obtained by separating it from the deep-frozen fluid medium). The planar product, e.g. the tissue web, is transported e.g. over a stationary plate (shoe). A moving screen is alternatively used as a supporting fabric which in turn slides e.g. over a suction box provided with a supporting perforated surface.

In industrial-scale tissue manufacturing, application preferably occurs after creping/drying on the yankee cylinder.

The method according to the invention is preferably performed within a by and large sealed system to enable the fluid medium and non-applied particles to be reprocessed or disposed of in an environmentally friendly manner.

Depending on the mesh width of the planar product to be treated and on the particle size (particle size distribution) of the frozen treatment composition, this composition more or less considerably penetrates into the planar product, particularly tissue. If the intention is to achieve an enrichment of the treatment composition on the outsides of a one-ply or multiply planar product, especially tissue, preference is given to selecting the particle size such that part of the particles, particularly more than 50%, much more preferably greater than 70%, optionally more than 80%, especially more than 95%, of the particles is greater than the average pore size (“mesh width”) of the planar product, particularly tissue, and is therefore enriched on the surface of the planar product.

Apart from the particle size distribution, it is possible to control the depth of penetration of the treatment composition by applying a vacuum at the side opposite the application side. The vacuum may also boost fixation of the particles, so far as they still move on the surface of the planar product, especially tissue. Application of a vacuum may come about during application in step (c), between step (c) and the heating step (d), during heating in step (d) or just after heating, the vacuum being preferably applied between steps (c) and (d) as regards fixation of the initially mobile particles, and during step (d) or just afterward as regards controlling the depth of penetration. Application of a vacuum preferably occurs by using a vacuum unit (e.g. vacuum box), over which the optionally multiply planar product, particularly tissue is guided, while applying a variable-intensity vacuum corresponding to the target setting for penetration. To support the planar product, particularly tissue, a supporting screen is preferably provided between the vacuum unit and the planar product, particularly the tissue. Upon application to a moving tissue web, use is preferably made of an endless belt that is frequently employed in the paper industry and which has a perforation; such an endless belt preferably moves at the same speed as the tissue web.

Lastly, for example by taking advantage of the treatment composition's wetting properties for the tissue and by utilizing gravity, it is also possible to control the depth of penetration by the temperature used in step (d) and for the duration of heat treatment.

Under certain conditions, e.g. if the planar product, particularly tissue, is to be guided past the application unit at a high speed, or if fine particles are to be applied, it may be advantageous to promote fixation of the particles on and/or within the optionally multiply planar product, especially tissue. This may for example be effected by electrostatically charging the deep-frozen particles of the treatment composition using an opposite charge to that of the planar product, particularly tissue, the effect of fixation being further heightened by additionally electrostatically charging the planar product, particularly tissue.

Particle fixation can also be promoted by applying a vacuum to the side opposite the application side. These fixing techniques may also be combined.

In step d (heating), the temperature of the treated planar product, particularly tissue, is allowed to rise to the melting point of the particles or a temperature above the melting point, preferably room temperature or a higher temperature.

This can be brought about without the assistance of special-purpose equipment in that the planar product, particularly tissue, with the treatment-composition particles applied thereto is slowly heated to room temperature.

A preferred heating option is to apply the particles to the planar product, particularly tissue, at a time when production conditions cause the product to exhibit an elevated temperature (usually up to 70° C.), e.g. in the case of tissue after creping/drying on the yankee cylinder.

In another preferred embodiment, heat is supplied in the form of heated air (e.g. by means of a fan), via an infrared radiation source or by using a heated roller to transport the tissue web treated with the frozen particles. This may be particularly necessary if the melting temperature of the treatment composition is above room temperature. The particles then melt and penetrate more or less considerably into the tissue web.

Treatment Composition

In accordance with the invention, the term “treatment composition” covers any substance or blends of substances generally referred to as treatment chemicals of a planar fiber-based web. In the case of tissue this term relates to chemicals normally applied to the tissue after the drying and creping step on the yankee cylinder.

Treatment chemicals may have an influence on physical properties, e.g. softness, particularly bulk softness, strength in the dry and wet states, rate of absorption of liquids, particularly that of water or oil, or the structural strength of the tissue/tissue product itself, and/or they may contribute to their varying use, e.g. in the field of skin care and protection, healthcare, etc. Adhesive compositions for laminating planar fibrous products, which are typically not intended to influence the physical or chemical properties of fiber-based planar webs, in particular tissue, are consequently not to be understood as “treatment compositions”.

The treatment composition may comprise a single treatment chemical or a blend of at least two treatment chemicals. This composition may also contain compounds that have no influence or only a slight influence on the properties of the treated planar product, particularly tissue, e.g. solvents (such as water and/or alcohol), auxiliary substances and/or additives. It may therefore be present e.g. as an aqueous solution or dispersion (e.g. suspension or emulsion) or comprise one or more treatment chemicals (water not included). Water may, however, also be an important active constituent of the treatment composition, particularly in cosmetic lotions intended to achieve a pleasant moist sensation on the skin. Water is then preferably used in combination with hygroscopic compounds such as the polyhydroxy compounds described below. Depending on the treatment composition's function, the proportion of optionally present solvents (including water) in the composition is preferably less than 60 wt. %, with greater preference on less than 30 wt. %, even greater preference on less than 10 wt. %, particularly less than 5 wt. %, each relative to the total weight of the composition.

If the intention is to apply several treatment chemicals in combination, they can also be separately converted into the frozen state and then mixed before application, optionally after a comminuting and/or classifying step.

The treatment chemical(s) may be selected from the following compound classes or compounds.

Agents for skin care and protection, so-called cosmetic lotions such as

moisturizers, such as substituents for the skin's natural moisturizing factor (NMF) that contain e.g. cleavage products of collagen, glycerol etc.;

skin care agents, e.g. long-chain fatty acid esters (like sorbitan fatty acid ester or Cetiol®), lanolin or derivatives thereof;

fragrances, e.g. natural, naturally identical or artificial perfumes; and/or

active cosmetic ingredients like D-panthenol or the active camomile ingredient α-bisabolol or agents exhibiting other functions, e.g.

strength-enhancing agents, particularly wet-strength agents like epichlorohydrin resins or crosslinked polyalkylene amines,

agents that promote the softness (e.g. bulk softness or surface softness) of the planar product, particularly the tissue; e.g. a polyhydroxy compound (e.g. ethylene glycol, propylene glycol, a liquid polyethylene glycol (derivative), a liquid polypropylene glycol (derivative) and/or glycerol), also quaternary ammonium compounds as described e.g. in U.S. Pat. Nos. 5,312,522 or 5,397,435 and the prior art cited therein, optionally in combination with the polyhydroxy compounds described in both these documents; or a poly(siloxane), particularly the (poly)siloxanes described in EP-A-347 153 and EP-A-347 154,

surfactants used e.g. as absorption rate control agents, e.g. long-chain quaternary ammonium compounds that may also exhibit softness-promoting action,

waxes, oils, and/or

inorganic or organic pigments or dyes.

Preferably the treatment composition comprises at least one of the following treatment chemicals: moisturizers, skin care agents, fragrances (aromatic principles), active medicinal and/or cosmetic ingredients, strength-enhancing agents, agents that promote tissue softness, and surfactants.

A preferred basic composition for improving softness, especially bulk softness, comprises the following recipe:

| | |
|---|---|
| glycerol: | 40-45% |
| propylene glycol: | 28-30% |
| linden extract: | 2.5-3.5% |
| water | up to 100% |

The total amount of nonvolatile treatment chemical(s) applied in the treated surface area of the planar product, particularly tissue, is preferably 0.01 to 50 wt. %, with greater preference on 0.5-45 wt. % and even greater preference on 0.75-40 wt. %, relative to the weight of the untreated oven-dried planar product, particularly tissue (oven-dried being understood in accordance with German standard DIN EN 20638). Even greater preference is given to values of 1-35 wt. %, particularly 2-30 wt. % (what is considered to be volatile is any component that volatilizes upon further processing of the planar product, especially tissue, e.g. solvent such as water, unless it is intended to remain in the composition, e.g. a cosmetic lotion.).

Heating the applied treatment composition may also be followed by a drying step, e.g. to remove the content of residual solvent in the treatment composition.

Fiber-based Planar Product

The present invention also relates to a fiber-based planar product that contains a treatment composition; this product can be obtained according to a method that comprises the steps described above. The term “fiber-based planar product”, as used here, stands for planar products made of fibers (especially fibers that contain cellulose, such as pulp), for example nonwovens or tissues, with tissue representing a particularly preferred embodiment.

The term “tissue” as defined by the present invention is understood as any kind of creped paper made from an aqueous dispersion and having a basis weight range of usually between 10 and 65 g/m². In accordance with the invention, the term “tissue” covers both

the entire range of raw creped paper, also known as “raw tissue”, particularly the range of dry-creped raw tissue paper, regardless of whether they are single-layer or multilayer,

and any single-layer or multilayer end products made of this creped raw paper.

“Raw tissue” is usually made as a one-ply tissue web in the tissue (paper) machine or as an optionally multiply (intermediate) product, e.g. in the form of multiply doubled webs or in the form of master rolls for further processing. The term “layers” refers to a change in chemical and/or physical properties within a tissue ply; such a change may be caused e.g. by a different fiber composition. In contrast to plies, layers usually cannot be separated from one another.

The final product is preferably

a cleaning wipe, e.g. wiping paper, a windscreen cleaning wipe, a cleaning wipe for industrial applications, a towel or a cleaning wipe for household use, e.g. kitchen paper;

a sanitary product, e.g. toilet paper (also moist);

a paper handkerchief (also moist);

a household towel, e.g. kitchen towels;

a towel;

a tissue for facial use, e.g. a makeup removal tissue (facial) or cosmetic tissue,

a serviette/napkin,

bed linen;

a garment, e.g. disposable apparel for hospitals or kitchen staff.

Particularly preferred tissue products are handkerchiefs, tissues for facial use, sanitary products (e.g. toilet paper) and towels in which the application of cosmetic treatment compositions and/or treatment compositions that convey softness (lotions) plays a part.

The term tissue paper must also be regarded independently of the fibrous raw material to be used, particularly irrespective of whether the fibrous raw material is made solely or mainly from natural pulps e.g. according to the sulfate or sulfite process, or is used in a mixture with chemothermomechanical wood pulps (e.g. CTMP, or HTCMP), or whether the fibrous raw material used comes from a secondary fiber refinement process and whether the fibrous raw material needed to make tissue therefore completely or partially comprises “recycled fibers”.

To distinguish from nonwovens, it should be noted that although the predominant use of natural, i.e. vegetable, pulp fibers broken up in a manner suitable for paper making is typical of tissue paper manufacturing, a proportional use by refinement of modified pulp fibers in a range of 10 to 50%, relative to the total weight of the fibers, or even a use of synthetic fibers suitable for paper making in an amount of 10 to 30% are covered by the aforementioned definition of the term “tissue”. It is analogously possible to apply the method beyond the field of paper making to corresponding fields in the nonwoven and textile sectors.

Upon application of the frozen particles, it is possible to start out e.g. from a multiply, usually two-ply to four-ply or multiply (doubled) master roll produced in a separate doubling machine. A plurality of one-ply tissue webs can alternatively be treated (one unwinding each) and then jointly rolled up into a multiply tissue product via a roll-up device. This produces the advantage that e.g. the inner plies can be treated with a treatment chemical other than that for the outer plies. For example, the inner plies of a four-ply end product can remain untreated, or can be treated with a strength-boosting agent, whereas the two outer plies were treated with a treatment chemical to improve surface softness. In principle, an extremely wide variety of combinations of differently treated tissue plies is conceivable.

In one embodiment, the tissue is a four-ply or three-ply doubled raw tissue for making handkerchiefs or facials, the tissue being made available in the form of master rolls for the application of a treatment agent in a processing machine suitable for this purpose. The processing machine comprises at least one unwinding device for the master rolls, a roll-up device for the product finished after application of a treatment agent, and an interposed applicator for applying the treatment agent.

Device

The present invention also relates to a device suitable for performing the method according to the invention, this device comprising the following features:

freezing means for freezing a treatment composition,

optionally means for producing particles from the frozen treatment composition, and

application means for applying the particles of the frozen treatment composition to a fiber-based planar product, e.g. tissue.

A device particularly suitable for performing the method according to claim 2 comprises at least the following features:

a) means for cooling a fluid medium,

b) means for bringing the cooled medium into contact with a treatment composition,

c) means for separating frozen particles of the treatment composition from the fluid medium, and

d) application means for applying the particles of the frozen treatment composition to the tissue.

It is in the unit (b) of the device that a frozen treatment composition is produced in the fluid medium. A solid having a specific size, e.g. in the form of coarse granules, as a deep-frozen substance or as a suspension of particles in the fluid medium can be produced here. The temperature within this unit preferably corresponds to those temperatures indicated above for the method, e.g. between −200 and −100° C.

The unit (c) comprises for example screens or classifiers that are preferably cooled.

The device contains application means as unit (d) in which the treatment-composition particles optionally separated from the fluid medium via screens can be applied to the tissue.

Technical heating means, such as those explained above, are not absolutely necessary, because the particles of the frozen treatment composition can also be adjusted to the temperature needed for thawing as a result of gradually heating to room temperature in the ambient air.

The device preferably also comprises means for comminuting a powder.

The device also preferably comprises means for classifying a powder.

In another preferred embodiment, the device comprises means for removing and reprocessing the fluid medium and/or the treatment composition.

The device according to the invention may further comprise means for pre-cooling the tissue web, and/or reheating/regulating the temperature of the tissue web. It may also include additional means for unwinding the tissue web, for transporting it and for re-reeling the finished product after application of the treatment composition.

To explain the aforementioned device according to the invention and its preferred embodiments, reference is also made to the description of the method according to the invention.

The method according to the invention is characterized by a low number of application steps—preferably a single such step—because treatment chemicals exhibiting chemical and/or physical properties that are not very compatible with one another (e.g. high viscosity/low viscosity, hydrophilic/hydrophobic) can also be systematically and evenly applied to the tissue by previous freezing.

The method according to the invention is also particularly suitable for the application of treatment chemicals that develop their action primarily at the surface of the planar product, especially tissue, e.g. cosmetic lotions or agents that promote surface softness such as the (poly)siloxanes described above. The reason for this is the ability to control the depth of penetration via a size distribution of the treatment composition's frozen particles, this particle size distribution being suitable for the mesh width of the tissue. Savings in the amount of treatment chemicals used can also be made in this way.

The systematic distribution of the treatment chemicals is also a feature of the products according to the invention, particularly tissue/tissue products. Tissue products in which the application has resulted in an enrichment of the treatment chemical(s) on one or both surfaces are particularly characterized by optimum development of their action.

1. A method of applying a treatment composition to a fiber-based planar product, said method comprising the steps of:
a) producing particles from a frozen treatment composition containing at least one treatment chemical,
c) applying the frozen particles of the treatment composition to the planar product,
d) heating the frozen particles of the treatment composition and the planar product, on which they were applied, to the melting point of the frozen treatment composition or to a higher temperature.

2. A method according to claim 1 comprising the steps of:
a)′ converting the treatment composition into the frozen state by bringing the treatment composition into contact with a fluid medium exhibiting a temperature at which the treatment composition is present in a frozen state, and
b) separating the frozen treatment composition from the fluid medium, optionally producing particles from the frozen treatment composition before or after step (b) if this composition has not accumulated in particle form upon conversion into the frozen state.

3. A method according to claim 1 or 2, wherein said treatment composition comprises at least one of the following treatment chemicals: moisturizers, skin care agents, fragrance, active medicinal and/or cosmetic ingredients, strength-enhancing agents, agents that promote tissue softness, and surfactants.

4. A method according to claim 1 or 2, wherein said treatment composition is applied to at least one outer surface of a fiber-based planar product having at least two plies.

5. A method according to any one of claims 1 to 4, wherein the amount of treatment chemical(s) applied is 0.01 to 50 wt. %, relative to the planar product (oven-dried).

6. A method according to claim 1 or 2, wherein coarse granules are produced in step (a) and said granules are comminuted between steps (a)/(a)′ and (c).

7. A method according to any one of claims 1, 2 or 6, wherein said frozen particles or said comminuted granules are classified into predetermined particle size classes.

8. A method according to any one of claims 2, 6 or 7, wherein said fluid medium is selected from a chlorofluorocarbon, fluorohydrocarbon, air or nitrogen each present in a gaseous state.

9. A method according to any one of claims 2, 6 or 7, wherein said fluid medium is selected from a chlorofluorocarbon, fluorohydrocarbon, air or nitrogen each present in a liquid state.

10. A method according to any one of the preceding claims, wherein application is effected by trickling the frozen particles onto the planar product.

11. A method according to any one of the preceding claims, wherein adhesion of the applied treatment composition to and within the planar product is promoted by application of a vacuum at that side of the planar product opposite the application side.

12. A method according to any one of the preceding claims, wherein said fiber-based planar product is a tissue.

13. A fiber-based planar product obtainable according to a method comprising the steps of at least one of claims 1 to 12.

14. A fiber-based planar product obtainable according to the method of claim 3, having a treatment composition comprising at least one of the following treatment chemicals: moisturizers, skin care agents, fragrance, active medicinal and/or cosmetic ingredients, strength-enhancing agents, agents that promote tissue softness, and surfactants.

15. A fiber-based planar product obtainable according to the method of claim 4, wherein said treatment composition was applied to at least one outer surface of a fiber-based planar product having at least two plies.

16. A planar product according to any one of claim 13, 14 or 15, said product being a tissue product.

17. A tissue product according to claim 16, wherein said treated tissue product is selected from a cleaning wipe, sanitary product, paper handkerchief, household towel, hand towel, tissue for facial use, a napkin/serviette, bed linen or a garment.
